(19) United States
(12) Patent Application Publication
(10) Pub. No.: US 2022/0340930 A1
(43) Pub. Date: Oct. 27, 2022

(54) METHODS OF OPTIMISING EXPRESSION AND DELIVERY OF MITOCHONDRIAL PROTEINS

(71) Applicant: CAMBRIDGE ENTERPRISE LIMITED, Cambridge (GB)

(72) Inventors: Michal Minczuk, Cambridge (GB); Payam Gammage, Cambridge (GB)

(21) Appl. No.: 17/441,165

(22) PCT Filed: Mar. 21, 2019
(86) PCT No.: PCT/GB2019/050808

§ 371 Date: Sep. 20, 2021

(51) Int. Cl. C12N 15/87; C12N 15/85

(52) U.S. Cl. CPC C12N 15/87; C12N 15/85; C12N 2750/14111

(57) ABSTRACT

The invention relates to methods for the simultaneous expression and delivery to mitochondria of two or more proteins using a single expression vector. Also described are the expression vectors and host cells comprising the vectors. Where the proteins are genome editing reagents, the invention also relates to the use of the expression vectors to alter levels of mitochondrial heteroplasmy and treat mitochondrial disorders.

### FIELD OF THE INVENTION

The invention relates to methods for the simultaneous expression and delivery to mitochondria of two or more proteins using a single expression vector. Also described are the expression vectors and host cells comprising the vectors. Where the proteins are genome editing reagents, the invention also relates to the use of the expression vectors to alter levels of mitochondrial heteroplasmy and treat mitochondrial disorders.

### BACKGROUND OF THE INVENTION

Mitochondrial diseases are a broad group of hereditary, multi-system disorders, a substantial portion of which are transmitted through mutations of mitochondrial DNA (mtDNA) with minimum prevalence of 1 in 5,000 adults. Human mtDNA is a small, double-stranded, multi-copy genome present at ˜100-10,000 copies per cell. In the disease state, mutated mtDNA often co-exists with wild-type mtDNA in heteroplasmy, and disease severity in conditions caused by heteroplasmic mtDNA mutations correlates with mutation load. A threshold effect, where >60% mutant mtDNA load must be exceeded before symptoms manifest, is a definitive feature of heteroplasmic mtDNA diseases.

Currently, there are no cures for mitochondrial diseases. For mitochondrial patients that can have children, genetic counselling and PGD (preimplantation genetic diagnosis) are currently the best options for preventing disease transmission. However, PGD can only reduce, not eliminate, the risk of transmitting the disease. Recently developed mitochondrial replacement techniques involve a series of manipulations of patient and donor oocytes, resulting in the generation of embryos carrying genetic material from three different origins. For these reasons, mitochondrial replacement techniques have raised biological, medical, and ethical concerns.

A novel alternative therapeutic approach to shift the heteroplasmic mtDNA ratio below the threshold of symptom manifestation has driven much research towards treatment of these incurable and essentially untreatable disorders. One such approach relies on generating site-specific double strand breaks in mtDNA using, for example, genome editing tools, such as mitochondrially targeted zinc finger-nucleases (mtZFNs). Because mammalian mitochondria lack efficient DNA double-strand break (DSB) repair pathways, selective introduction of DSBs into mutant mtDNA leads to rapid degradation of these molecules. As mtDNA copy number is maintained at a cell type-specific steady-state level, selective elimination of mutant mtDNA stimulates replication of the remaining mtDNA pool, eliciting a shift in the heteroplasmic ratio.

We have recently developed an in vivo experimental gene therapy for heteroplasmic mitochondrial diseases. We use genome editing with mtZFNs in a mouse model to specifically eliminate mutant, pathogenic mtDNA, allowing WT mtDNA to take its place.

Elimination (or at least a reduction in the amount) of mutant mtDNA is coupled to a reversion of molecular and biochemical phenotypes following delivery of mtZFNs by systemically administered adeno-associated virus (AAV). This sequence-tailored mitochondrial gene editing approach offers the prospect of a cure for thus far incurable mitochondrial diseases.

To date, mtZFN-based therapy has depended on delivering two distinct ZFN monomers to the same cell using two separate vectors, which requires double transfection/injection relying on heavy dosage for efficacy. The mtZFN gene editing approaches, like many therapies, have to find a therapeutic balance between being too aggressive in dosing (e.g. causing excessive mtDNA depletion) or too weak (not achieving mutant mtDNA reduction). Being able to balance the efficacy vs. toxicity trade-off is key to making mtZFN therapies a viable treatment option. The present invention addresses this need.

Aside from the treatment of mitochondrial diseases, there also exists a need to improve on a general level the efficiency of delivery of two or more agents, in particular therapeutic or diagnostic agents, to intracellular organelles, such as mitochondria. Again, a balance needs to be struck between efficacy and toxicity. The present invention also addresses this need.

### SUMMARY OF THE INVENTION

By this invention we refine mitochondrial (mt) genome editing, such as mtZFN therapies by placing, as an example, both ZFN monomers in the same AAV virion. This approach allows for the production of only a single mtZFN-AAV product for therapeutic applications. This approach should significantly minimize regulatory (approval of one rather than two products) and production cost issues arising from placing mt nucleases as monomers in separate AAVs. Importantly, it also allows for much lower doses of virus to be administered whilst retaining efficacy, as co-infection of the same cell (as required in the monomer approach) is no longer a concern.

Accordingly, in a first aspect of the invention, there is provided a nucleic acid molecule for simultaneous expression and delivery to the mitochondria of at least two proteins, the nucleic acid molecule comprising a first nucleic acid sequence encoding a first mitochondrial localisation signal (MLS) and a first protein and a second nucleic acid sequence encoding a second mitochondrial localisation signal and a second protein, wherein the first and second nucleic acid sequence are separated by at least one ribosomal skipping sequence and wherein the first and second nucleic acid sequences are operably linked to a regulatory sequence.

In a preferred embodiment, the first and second nucleic acid sequences encode a first and second protein, wherein the percent sequence identity of the amino acid sequence is higher than the percent sequence identity of the nucleic acid sequence between the first and second protein.

In a more preferred embodiment, the first and second nucleic acid sequences encode proteins with a minimum of 70 to 90% amino acid sequence identity and a maximum of 55 to 70% nucleic acid sequence identity. In a further preferred embodiment, the first and second nucleic acid sequences encode proteins with a minimum of 80 to 90% amino acid sequence identity and a maximum of 60 to 70% nucleic acid sequence identity. In a particular embodiment, the first and second nucleic acid sequences encode proteins with a minimum of 82% amino acid sequence identity and a maximum of 63% nucleic acid sequence identity.

In an alternative or additional embodiment, the nucleic acid sequence of the first and second proteins do not have a stretch of sequence identity (also referred to herein as “homology”) longer than 1 to 40 bp, more preferably 6 to 30 bp, more preferably 6 to 20 bp, preferably 6 to 15 bp, and even more preferably 9 bp.

In another aspect of the invention, there is provided a nucleic acid molecule for simultaneous expression and delivery to the mitochondria of at least two proteins, the nucleic acid molecule comprising a first nucleic acid sequence encoding a first mitochondrial localisation signal and a first protein and a second nucleic acid sequence encoding a second mitochondrial localisation signal and a second protein, wherein the first and second nucleic acid sequences encode proteins with a minimum of 70 to 90% amino acid sequence identity and a maximum of 50 to 70% nucleic acid sequence identity, and wherein the first and second nucleic acid sequence are separated by at least one ribosomal skipping sequence and wherein the first and second nucleic acid sequences are operably linked to a regulatory sequence.

In one embodiment, the second mitochondrial localisation signal comprises one or more N-terminal amino acids that mask the mitochondrial localisation signal. In a preferred embodiment, the second mitochondrial localisation signal comprises an N-terminal proline.

In a preferred embodiment, the first and second proteins comprise a DNA-binding polypeptide and nuclease.

In another embodiment, the ribosomal skipping sequence is a nucleic acid sequence encoding a 2A peptide. Preferably, the 2A peptide is selected from T2A, P2A, E2A and F2A.

In one embodiment, the DNA-binding polypeptide is a zinc finger DNA binding domain. In another preferred embodiment, the nuclease is FokI.

In a further embodiment, the first and second nucleic sequences further encode a nuclear export signal.

In another embodiment, the nucleic acid molecule is contained within a vector. Preferably, the vector is a viral or non-viral vector. More preferably, the viral vector is an adeno-associated virus.

In one embodiment, the regulatory sequence is a promoter.

In another aspect of the invention, there is provided a host cell comprising a nucleic acid molecule as described herein.

In a further aspect of the invention, there is provided a nucleic acid molecule as described herein for use as a medicament.

In another aspect of the invention there is provided a nucleic acid molecule as described herein for use in the treatment of a mitochondrial disease.

In a further aspect of the invention, there is provided a method of therapy, the method comprising administering the nucleic acid molecule as described herein to a patient or individual in need thereof.

In yet a further aspect of the invention, there is provided a method of treating a mitochondrial disease, the method comprising administering the nucleic acid molecule as described herein to a patient or individual in need thereof.

In another aspect of the invention, there is provided a method of changing mitochondrial DNA heteroplasmy, the method comprising administering the nucleic acid molecule as described herein to a target cell or tissue.

In a further aspect of the invention, there is provided a method of introducing at least one single-strand and/or double-strand break into mitochondrial DNA, the method comprising administering the nucleic acid molecule as described herein to a target cell or tissue.

In a final aspect of the invention, there is provided a method for simultaneous expression and delivery to the mitochondria of at least two proteins, the method comprising administering the nucleic acid molecule as described herein to a target cell or tissue. Preferably, the expression and/or import of the first protein in the mitochondria is higher than the expression and/or import of the second protein in the mitochondria.

### DESCRIPTION OF THE FIGURES

The invention is further described in the following non-limiting figures:

FIG. 1 shows evidence for delivery and effective mtDNA heteroplasmy shift of mtZFN monomers as a single ORF (open reading frame) into cultured cells.
(A) Schematic of the general workflow for experiments that involve transient transfection of heteroplasmic cells with plasmids co-expressing mtZFN monomers and fluorescent marker proteins, FACS-based selection of cells expressing both mtZFN monomers and phenotypic evaluation of mtZFN-treated cells.
(B) Refined strategy that involve transient transfection of heteroplasmic cells with a single plasmid co-expressing mtZFN monomers separated by a X2A peptide.
(C) Detailed schematic of strategy for selective degradation of m.8993T>G mtDNA using mtZFN. Conventional dimeric, engineered mtZFN are directed to sequence adjacent to (COMPa, green) or including (NARPd, red) the mutated base position. Both monomers should bind the substrate only when the indicated nucleotide is mutated and not to the wild-type sequence. DNA double strand breaks should only be introduced into the mutant mtDNA molecule, leading to a shift in heteroplasmy.
(D) Variants of x2A peptide tested in in-vitro preliminary experiments with m.8993T>G cybrids.
(E) Western blot showing expression of mtZFN monomers, separated by different x2A peptides in m.8993T>G cybrids.
(F) Shift in m.8993T>G heteroplasmy by mtZFN separated by different x2A peptides. RFLP analysis of last-cycle hot PCR products (mtDNA nt positions 8339-9334) amplified from total DNA samples of 143B cells harboring indicated levels of m.8993T>G, obtained by treatment with mtZFNs. Wild-type cells and 100% m.8993T>G cybrids were used as controls.

FIG. 2 shows evidence for delivery and improved in vivo action of mtZFN deliver as a T2A AAV compared to two AAV virion approach.
(A) The mtZFN monomers are separated by a T2A peptide, allowing for expression of the two protein constructs from a single ORF. Schematics of the AAV-mtZFN-T2A construct: LRT (L/R)—Inverted Terminal Repeat; CMV—cytomegalovirus promoter; WPRE—Woodchuck Hepatitis Virus Posttranscriptional Regulatory Element; BGH pA—RNA polyadenylation signal from bovine growth hormone mRNA. Mitochondrial targeting of mtZFN is facilitated by a 49-amino acid-long MLS from subunit F1β of human mitochondrial ATP synthase. NES, nuclear export signal; ZFP, zinc finger peptide; HA, haemagglutinin tag.
(B) Scheme of in vivo experiments. MTM25 and WTM1 are encoded in separate AAV genomes (red and blue hexagon) or within a single AAV separated by T2A (purple hexagon), then administered by tail-vein (TV) injection. Animals are sacrificed at 65 days post-injection.
(C) Pyrosequencing of m.5024C>T heteroplasmy from ear [E] and heart [H] total DNA. Change (Δ) in m.5024C>T is plotted. Black triangle indicates increasing doses of AAV. For separate 2x monomer approach the doses are: 5*10¹¹, 1*101² and 1*10¹³ (outline, solid colour, striped bar respectively). For the T2A approach equivalent doses are: 2.5*10¹¹, 5*10¹¹ and 5*10¹² (outline, solid colour, striped bar respectively). Animal number: n=20 (vehicle), n=4 (all other conditions). Error bars indicate the s.e.m. Statistical analysis performed: two-tailed Student's t-test. Vehicle/intermediate dose p<0.00001, Vehicle/high dose p<0.00001. Measure of center is the mean.
(D) Assessment of mtDNA copy number by quantitative PCR in the conditions as per (C). n=20 (vehicle) and n=4 (all other conditions). The centre line is the mean and the error bars indicate the s.e.m. Statistical analysis was performed using the two-tailed Student's t-test; P=0.007931. Central black line indicates the mean. ** indicates P<0.01, *** indicates P<0.001.

FIG. 3 shows Time-dependent heteroplasmy shifting activity of AAV-delivered mtZFNs in the heart. MTM25 and WTM1 encoded in separate AAV genomes were administered by tail-vein (TV) injection at 5*10¹² vg AAV titer. Animals are sacrificed at 65 or 130 days post-injection. The graph shows pyrosequencing of m.5024C>T heteroplasmy from ear [E] and heart [H] total DNA. Change (Δ) in m.5024C>T is plotted. Black triangle indicates increasing time of 65 or 130 days post injection. Animal number: n=20 (vehicle), n=4 (65 days), n=4 (130 days). Error bars indicate the s.e.m. Statistical analysis performed: two-tailed Student's t-test. Vehicle/intermediate dose p<0.00001, Vehicle/high dose p<0.00001. Measure of center is the mean.

FIG. 4 is a comparison of the dose needed to achieve effective heteroplasmy when mtZFN monomers are delivered in separate vectors (“2x monomer”) compared to the single vector (“1XT2A”) of the present invention.

*The doses are adjusted to reflect the simultaneous delivery of both mtZFN monomers within every transduced cell and to allow a direct comparison between the separate 2x AAV monomer approach and the monomer1-T2A-monomer2-AAV method.

According to this: 5*10¹¹ separate 2x AAV monomer dose is an equivalent of 2.5*10¹¹ T2A dose.

**** copy number depletion observed.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of microbiology, tissue culture, molecular biology, chemistry, biochemistry, recombinant DNA technology, and bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

In one aspect of the invention, there is provided a nucleic acid molecule for simultaneous expression and delivery to the mitochondria of two or more proteins, the nucleic acid molecule comprising a first nucleic acid sequence encoding a first mitochondrial localisation signal and a first protein and a second nucleic acid sequence encoding a second mitochondrial localisation signal and a second protein, wherein the first and second nucleic acid sequence are separated by at least one ribosomal skipping sequence and wherein the first and second nucleic acid sequences are operably linked to a regulatory sequence.

In one embodiment, the two proteins can be any two proteins where simultaneous expression and delivery to the mitochondria is desired.

The proteins may be the same or different. That is, the nucleic acid molecule can be used to simultaneously express and deliver to the mitochondria two or more of the same protein or different proteins. As an example, the former may be useful to deliver a target protein and a marker (such as a fluorescent marker, for example) for that target.

The latter may be useful, for example, when the two proteins depend on each other for function or alternatively, act synergistically. For example, the proteins may be monomers or subunits of a protein complex or oligomer. In one example, the proteins may be zinc finger nuclease (ZFN) monomers. As explained below, in one embodiment, the protein comprises or consists of a DNA-binding domain and a DNA-cleavage domain, or nuclease.

In one embodiment, where the first and second nucleic acid sequences encode a first and second protein, the percent sequence identity of the amino acid sequence is preferably higher than the percent sequence identity of the nucleic acid sequence between the first and second protein.

In a particular embodiment, the amino acid sequence of the proteins are at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical but the nucleotide sequence of the proteins is less than the level of sequence identity at the amino acid level. For example, in one embodiment, the nucleotide sequence of the proteins are a maximum of 55%, 56%, 57%, 58%, 59%, 60%, 61%, 62%, 63%, 64%, 65%, 66%, 67%, 68%, 69%, 70%, 71%, 72%, 73%, 74% or 75% identical. In a more preferred embodiment, the first and second nucleic acid sequences encode proteins with a minimum of 60 to 99% amino acid sequence identity and a maximum of 55 to 70% nucleic acid sequence identity. In a further preferred embodiment, the first and second nucleic acid sequences encode proteins with a minimum of 70 to 90% amino acid sequence identity, more preferably 80 to 90% amino acid sequence identity and a maximum of 55 to 60% nucleic acid sequence identity. In a particular embodiment, the first and second nucleic acid sequences encode proteins with a minimum of 82% amino acid sequence identity and a maximum of 63% nucleic acid sequence identity. In another example, the first and second nucleic acid sequences encode proteins with 100% amino acid sequence identity and a maximum of 57% nucleic acid sequence identity.

In an alternative or additional embodiment, the nucleic acid sequence of the first and second proteins do not have a stretch of sequence identity (also referred to herein as “homology”) longer than 6 to 40 bp, more preferably 6 to 30 bp, more preferably 6 to 20 bp, preferably 6 to 15 bp, and even more preferably 9 bp.

By “stretch of homologous nucleotides” is meant nucleotides that are at least 98%, 99% or 100% identical.

Accordingly, the nucleic acid sequence encoding at least one of the proteins contain one or more modifications or differences from the nucleic acid sequence encoding the other protein, and these preferably lead to a change at the codon level—i.e. a change in codon bias. This means that although the two nucleic acid sequences of the proteins differ as defined above, due to the degeneracy of the genetic codon, the resulting amino acid sequence has a high level of sequence identity, again as defined above. This is referred to herein as “recoding”.

In one embodiment, only one of the proteins is recoded. In another embodiment, both proteins are recoded. In one embodiment the nucleic acid sequence of the mitochondrial localisation signal (MLS) and/or the protein are recoded, as defined herein. In an alternative embodiment however, instead of recoding, the first and second nucleotide sequences different mitochondrial localisation signals are used, as discussed below.

We have found that recoding the first and/or second nucleic acid sequence significantly increases the viability of the nucleic acid molecule, particularly when the nucleic acid molecule is a vector, and more particularly a viral vector such as an adeno-associated viral vector. In particular, we have found that when the nucleic acid molecule is used to express two highly similar proteins (i.e. a high level of sequence identity at the nucleotide level) we observed large-scale deletions within the vector. We have further found that these large-scale deletions are caused by recombination of the homologous protein-coding genes/nucleotide sequences. That this was the cause of the large-scale deletions was unexpected, particularly as no such deletions were observed when the vector was a plasmid. However, we have found that recoding of the first and/or second nucleic acid sequence avoided the large-scale deletions and led to high titres of viral particles with full length, faithfully encoded nucleic acid sequences.

Accordingly, in a further aspect of the invention, there is provided a nucleic acid molecule for simultaneous expression and delivery to the mitochondria of two or more proteins, the nucleic acid molecule comprising a first nucleic acid sequence encoding a first mitochondrial localisation signal and a first protein and a second nucleic acid sequence encoding a second mitochondrial localisation signal and a second protein, wherein the first and second nucleic acid sequences are separated by at least one ribosomal skipping sequence and wherein the first and second nucleic acid sequences are operably linked to a regulatory sequence.

In one embodiment, the first nucleic acid sequence encodes a N-terminal mitochondrial localisation signal fused to a first protein. Similarly, the second nucleic acid sequence encodes a N-terminal mitochondrial localisation signal fused to the second protein.

By “mitochondrial localisation signal” (MLS) is meant a peptide sequence that directs a protein to the mitochondria. Typically a MLS is 10-70 amino acids in length and consists of an alternating pattern of hydrophobic and positively charged amino acids that form an amphipathic helix. Examples of MLS sequences include the following or functional variants thereof:

(SEQ ID NO: 1)
MLGFVGRVAAAPASGALRRLTPSAS
LPPAQLLLRAAPTAVHPVRDYAAQ;
(SEQ ID NO: 35)
MSVLTPLLLRGLTGSARRLPVPRAK
(SEQ ID NO: 36)
MQTAGALFISPALIRCCTRGLIRPV
SASFLNSPVNSSKQPSYSNFPLQVA
RREFQTSWVSR
(SEQ ID NO: 37)
MLGFVGRVAAAPASGALRRLTPSAS
LPPAQLLLRAAPTAVHPVRDYA
(SEQ ID NO: 38)
MLRAAARFGPRLGRRLL
(SEQ ID NO: 39)
RSGIIKASRVLYRQM

Other MLS sequences would be known to the skilled person, and include the sequences described in Minczuk M et al., 2006, which is incorporated herein by reference.

In one embodiment, the sequence of the MLS of the first and second nucleic acid sequence are the same. In an alternative embodiment, the sequence of the MLS of the first protein and the sequence of the MLS of the second protein are different.

In one embodiment, the MLS sequence comprises an additional amino acid residue, preferably an N-terminal additional amino acid that “masks” the MLS—that is, it slows the rate of import of the targeted sequence to the mitochondria (compared to a sequence that lacks this additional amino acid). In a preferred embodiment, the additional amino acid residue is a proline residue.

By “masks” is meant that the localisation signal is partially disabled. That is, the protein is not localised to the mitochondria as efficiently as a protein that carries a MLS without the additional amino acid residue. This is exemplified in FIG. 1(e). FIG. 1(e) is a western blot showing the expression of the mitochondrial zinc finger nucleases (ZFN), NARPd and COMPa. COMPa is the second protein as used herein and comprises an additional N-terminal proline on its MLS sequence (as a result of processing of the ribosomal skipping sequence). As the MLS is cleaved upon import into mitochondria the imported protein is represented by the lower band, while the upper band represents the protein with the MLS, which has not been taken up into the mitochondria. As can be seen from FIG. 1(e), protein 1 (NARPd) is imported more readily than protein 2 (COMPa). The different rate of import is implied by the different amount of import, which is caused by masking of the MLS of protein 2.

This has significant benefit, especially in this context, where the slower import of the second ZFN monomer reduces the possibility of off-target effects. This is shown in FIGS. 2c and 2d. FIG. 2c shows that at a high dose of the two AAV virions there is a significant shift in heteroplasmy but that this same dose is also accompanied by a significant decrease in copy number (FIG. 2d). In comparison, the T2A AAV virion also significantly shifts heteroplasmy without affecting copy number to a statistically significant extent (FIG. 2d).

In one embodiment, the first and/or second nucleic acid sequences encode a protein, where the protein comprises or consists of a DNA-binding domain and a DNA-cleavage domain, or nuclease. In one embodiment, the protein can be used for targeted genome modification or targeted genome editing of the mitochondrial DNA.

Targeted genome modification or targeted genome editing is a genome engineering technique that uses targeted DNA double-strand breaks (DSBs) to stimulate genome editing through homologous recombination (HR)-mediated recombination events. However, as explained above, when genome editing is used in the context of mitochondria, the absence of any efficient repair pathways means that the introduction of double-stranded breaks causes degradation of the targeted mitochondrial genome. Where the target of genome editing is a pathogenic mtDNA mutation degradation of the pathogenic mtDNA genome alters or causes a shift in the mitochondrial heteroplasmy towards the wild-type state. These approaches therefore have significant value in the treatment of mitochondrial diseases.

To achieve effective genome editing via introduction of site-specific DNA DSBs, four major classes of customisable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of FokI to direct nucleolytic activity toward specific genomic loci.

Zinc-finger nucleases contain two domains; a DNA-binding domain and a DNA-cleavage domain. The DNA-binding domain typically contains three to six zinc finger repeats, able to recognise between nine and eighteen base pairs in the target DNA sequence. The DNA-cleavage domain is reliant on the DNA-binding domain as it has no sequence specificity. Most often used as the DNA-cleavage domain is the type II restriction endonuclease FokI, which requires dimerization for cleavage and so pairs of zinc finger nucleases must be designed to target non-palindromic DNA sequences. A linker sequence of 6-15 bp is required between the binding and cleavage domains.

In cultured cells, constructs expressing zinc finger-nucleases use promoters optimised for the cell type and are introduced using a vector by transfection. Embryo injection was first reported by Beumer et al and showed that delivery of zinc finger-nucleases could be achieved through injection of zinc finger nuclease mRNAs and donor DNA into embryos. This breakthrough led to the use of zinc finger nucleases to generate viable adults carrying germline mutations when grown from treated embryos in zebrafish, rats, mice, sea urchin, silkworm and Drosophila.

By comparison, TAL effectors enter the nucleus following delivery through the bacterial type III secretion system and bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD that determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the Fokl nuclease to create a TAL effector nuclease (TALEN) which makes targeted DNA double-strand breaks (DSBs) in vivo for genome editing. Cermak T et al. describes a set of customized plasmids that can be used with the Golden Gate cloning method to assemble multiple DNA fragments. As described therein, the Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct. Accordingly, using techniques known in the art it is possible to design a TAL effector that targets a pathogenic mtDNA sequence as described herein.

Accordingly, in one embodiment, the DNA-binding domain is a zinc finger DNA-binding domain. As discussed above, typically such a DNA-binding domain contains between three and six individual zinc finger repeats and can each recognise between nine and eighteen base pairs in the target sequence.

In one example, the target sequence is a mtDNA polymorphism (point mutation), and in particular, a pathogenic mtDNA polymorphism. In a further example, the DNA-binding domain targets one of the following pathogenic mtDNA polymorphisms: m.3243A>G, m.3271T>C, m.8344A>G, m.8356T>C, m.8993T>C/G, m.8363G>A, m.11778G>A, m.3460G>A, m.3394T>C, m,3291T>C, m.3303C>T, m.3302A>G, m.3250T>C, m.3256C>T, m.3251A>G, m. 3252A>G and m.3260A>G. In an alternative example, the polymorphism may be the deletion of one or more nucleotides. As an example, the deletion may be selected from m.del_8469:13447 (also known as the ‘common deletion’), m.del_8482:13460, m.del_12112:14412, m.del_11232:13980, m.del_8648:16085, m.del_547:4443, m.del_7841:13905 and m.del_8271:8281.

In one example, the protein sequence of the zinc finger DNA-binding domain is selected from SEQ ID NO: 7 and 9 or a functional variant thereof. In a further example, the nucleic acid sequence of the zinc finger DNA binding domain is selected from SEQ ID NO: 18 and 20 or a functional variant thereof.

In a particular embodiment, the first nucleic acid sequence encodes a protein that comprises a DNA binding domain as defined in SEQ ID NO: 7 or a functional variant thereof and the second nucleic acid sequence encodes a protein that comprises a DNA-binding domain as defined in SEQ ID NO: 9 or a functional variant thereof.

In an alternative embodiment, the DNA-binding domain may be a TAL effector. By “TAL effector” (transcription activator-like (TAL) effector) or TALE is meant a protein sequence that can bind a mitochondrial DNA target sequence and that can be fused to the cleavage domain of an nuclease, such as FokI to create TAL effector nucleases or TALENS or meganucleases to create megaTALs. A TALE protein is composed of a central domain that is responsible for DNA binding, a nuclear-localisation signal and a domain that activates target gene transcription. The DNA-binding domain consists of monomers and each monomer can bind one nucleotide in the target nucleotide sequence. Monomers are tandem repeats of 33-35 amino acids, of which the two amino acids located at positions 12 and 13 are highly variable (repeat variable diresidue, RVD). It is the RVDs that are responsible for the recognition of a single specific nucleotide. HD targets cytosine; NI targets adenine, NG targets thymine and NN targets guanine (although NN can also bind to adenine with lower specificity). Where the first and/or second nucleic acid sequence encode a TALEN, the nucleic acid molecule is preferably not an AAV, as described below. The nucleic acid molecule may be a plasmid.

In a further embodiment, the first nucleic acid sequence may encode a ZFN and the second nucleic acid sequence may encode a TALEN, as described above—or vice versa.

By “nuclease” is meant any enzyme that comprises a DNA cleavage domain. In other words, an enzyme that can cleave at least one DNA strand (called a nickase) or preferably both DNA strands (nuclease). That is, the nuclease can create a single or double-stranded break in the mitochondrial DNA sequence—the former are created by inactivating the catalytic activity of one ZFN monomer in the ZFN dimer that is required for double-strand cleavage. In one embodiment, the nuclease is an endonuclease, preferably a type II restriction endonuclease, and more preferably a type IIS restriction nuclease. Examples of suitable nucleases include FokI, AcuI, AlwI, BaeI, BbsI, BbsI-HF, BbvI, BccI, BceAI, BcgI, BciVI, BcoDI, BfuAI, BmrI, BpuEI, BsaI, BsaI-HF, BsaXI, BseRI, BsgI, Bsm AI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MmeI, MnlI, NmeAllI, PleI, SapI and SfaNI. In a preferred embodiment, the nuclease is FokI.

In a particular embodiment, the first nucleic acid sequence encodes a protein that comprises a DNA cleavage domain or nuclease as defined in SEQ ID NO: 8 or a functional variant thereof and the second nucleic acid sequence encodes a protein that comprises a DNA cleavage domain or nuclease as defined in SEQ ID NO: 10 or a functional variant thereof.

As described above, the first and second nucleic acid sequences are separated by at least one ribosomal skipping sequence.

A “ribosomal skipping sequence” is a sequence, which when translated in the nascent peptide chain prevents the ribosome from creating the peptide bond with the next proline. As a result, translation is stopped, the nascent polypeptide is released and translation is re-initiated to produce a second polypeptide. This mechanism results in apparent co-translational cleavage of the polyprotein. As such, a ribosomal skipping sequence allows two proteins to be expressed as individual proteins from a single mRNA molecule.

In one embodiment, the ribosomal skipping sequence is a 2A-like peptide. Examples of 2A-like peptides include:

T2A:
(SEQ ID NO: 27)
EGRGSLLTCGDVEENPGP
T2A*:
(SEQ ID NO: 28)
GSGEGRGSLLTCGDVEENPGP
P2A:
(SEQ ID NO: 29)
GATNFSLLKQAGDVEENPGP
P2A*:
(SEQ ID NO: 30)
GSGATNFSLLKQAGDVEENPGP
E2A:
(SEQ ID NO: 31)
QCTNYALLKLAGDV ESNPGP
E2A*:
(SEQ ID NO: 32)
GSGQCTNYALLKLAGDV ESNPGP
F2A:
(SEQ ID NO: 33)
VKQTLNFDLLKLAGDVESNPGP
F2A*:
(SEQ ID NO: 34)
GSGVKQTLNFDLLKLAGDVESNPGP

Accordingly, in one embodiment, the 2A-like peptide is selected from one of the above 2A-like sequences or a functional variant thereof.

The use of a P2A peptide results in the addition of a C-terminal ribosomal skipping sequence (or the majority of such a sequence) to the first polypeptide chain (i.e. the the protein encoded by the first nucleic acid sequence), and an N-terminal proline to the next polypeptide (i.e. the protein encoded by the second nucleic acid sequence). As explained above, we have found that the N-terminal proline on the second MLS masks the signal sequence and slows the rate of transport of the second protein into the mitochondria.

In an alternative embodiment, the ribosomal skipping sequence is a tRNA sequence. tRNA sequences may be used to allow multiple RNAs to be produced from a single engineered polycistronic gene consisting of tandemly arrayed tRNA-RNA units. After the polycistronic gene is transcribed by endogenous transcriptional machinery, the endonucleases RNAse P and RNAse Z (or RNAse E in bacterium) recognise and specifically cleave the tRNAs at specific sites at the 3′ and 5′ ends, releasing mature RNAs and tRNAs. Advantageously, tRNAs and their processing system are virtually conserved in all living organisms and therefore this method can be used in all known species. In another alternative, the ribosomal skipping sequence may be an internal ribosome entry site or IRES. The sequences of suitable IRESs would be well known to the skilled person.

In an alternative embodiment, instead of a ribosomal skipping sequence, the first and second nucleic acid sequences are separated by at least one sequence that allows self-cleavage/self-processing of the first and second nucleic acid sequences upon transcription. For example, the first and second nucleic acid sequences may be separated by at least one nucleic acid sequence that encodes a ribozyme enzyme. Once transcribed, the primary transcripts will undergo self-catalysed cleavage to generate two MLS-proteins.

In one example, the ribozyme enzyme may be selected from a Hammerhead (HH) ribozyme unit and/or a hepatitis delta virus (HDV) ribozyme unit. In one embodiment, the sequence of the HH (Hammerhead) ribozyme comprises the following sequence:

CTGATGAGTCCGTGAGGACGAAACGAGTAAGCTCGTC (SEQ ID NO: 40) or a variant thereof as described. In a further embodiment, the sequence of hepatitis delta virus (HDV) ribozyme is:
GGCCGGCATGGTCCCAGCCTCCTCGCTGGCGCCGGCTGGGCAACATGCTTCGG CATGGCGAATGGGAC (SEQ ID NO: 41) or a variant thereof as described herein.

Where such alternative ribosomal skipping sequences or ribozymes are used, for example, the nucleic acid molecule may further comprise one or more Kozak sequences ((gcc)gccRccAUGG) (SEQ ID NO: 42) or a functional variant thereof to serve as a translational start site. Preferably such a sequence would be between the first and second nucleic acid sequences.

In another embodiment, the first and/or second nucleic acid sequence further encode a nuclear export signal (NES). Typically a NES sequence is a short amino acid sequence of four hydrophobic residues that targets a nascent polypeptide for export from the nucleus into the cytoplasm through the nuclear pore complex using nuclear transport. In one example, the NES has an amino acid sequence as defined in SEQ ID NO: 4 and a nucleic acid sequence defined in SEQ ID NO: 15 or functional variants thereof.

In a further embodiment, the nucleic acid molecule comprises at least one, preferably two inverted terminal repeats or LTRs. Preferably, the nucleic acid molecule comprises a 5′ LTR and a 3′ LTR. The 5′ LTR acts as an RNA pol II promoter. The 3′LTR acts as a terminator sequence, marking the end of the operon and causing transcription to stop.

In a preferred embodiment, the first and second nucleic acid sequences are operably linked to at least one regulatory sequence. More preferably, the first and second nucleic acid sequences are operably linked to one regulatory sequence.

The term “operably linked” as used throughout refers to a functional linkage between the regulatory sequence and the first and/or second nucleic acid sequence such that the regulatory sequence is able to initiate transcription of the first and/or second nucleic acid sequence.

According to all aspects of the invention, the term “regulatory sequence” is used interchangeably herein with “promoter” and all terms are to be taken in a broad context to refer to regulatory nucleic acid sequences capable of effecting expression of the sequences to which they are ligated. The term “regulatory sequence” also encompasses a synthetic fusion molecule or derivative that confers, activates or enhances expression of a nucleic acid molecule in a cell, tissue or organ.

The term “promoter” typically refers to a nucleic acid control sequence located upstream from the transcriptional start of a gene and which is involved in the binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences.

In one embodiment, the promoter may be a constitutive promoter. A “constitutive promoter” refers to a promoter that is transcriptionally active during most, but not necessarily all, phases of growth and development and under most environmental conditions, in at least one cell, tissue or organ. Examples of constitutive promoters include the CMV, CAG, Rosa26, ubiquitin, actin, tubulin, GAPDH, PGK, SV40 and EF1A promoter.

In an alternative embodiment, the promoter may be a tissue-specific promoter. A tissue specific promoter is a transcriptional control element that is only active in particular cells or tissues.

For the identification of functionally equivalent promoters, the promoter strength and/or expression pattern of a candidate promoter may be analysed for example by operably linking the promoter to a reporter gene and assaying the expression level and pattern of the reporter gene in various tissue. Suitable well-known reporter genes are known to the skilled person and include for example beta-glucuronidase or beta-galactosidase.

In one specific embodiment, the nucleic acid molecule may comprise a nucleic acid sequence as defined in SEQ ID NO: 26 or a functional variant thereof.

The term “variant” or “functional variant” as used throughout with reference to any of SEQ ID NOs refers to a variant nucleotide or protein sequence that retains the biological function of the full non-variant sequence. A functional variant also comprises a variant that has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence or ribonucleic acid sequence that result in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

As used in any aspect of the invention described throughout a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

In a preferred embodiment, the nucleic acid molecule is a vector or is contained within a vector.

In one embodiment, the vector is a viral vector. More preferably the viral vector is selected from adenoviruses, adeno-associated viruses (AAV), alphaviruses, flaviviruses, herpes simplex viruses (HSV), measles viruses, rhabdoviruses, retroviruses, lentiviruses, Newcastle disease virus (NDV), poxviruses, picornaviruses and hybrids thereof. In one embodiment, the vector is an adeno-associated virus (AAV) or AAV variant. In a further preferred embodiment, the AAV may be selected from serotype 1, 2, 3, 4, 5, 6, 7, 8, 9, 10 or 11. The skilled person would understand that serotypes differ in their tropism and as such the selection of the target tissue will depend on the target tissue to be infected. For example, where the target tissue is the heart, the AAV may be selected from serotypes 1, 8 and 9, and most preferably is AAV9. In another example, where the target tissue is the brain or CNS, the AAV may be selected from serotypes 1, 2, 4, 5, 8 and 9. Similarly, when the target tissue is muscle, the AAV may be selected from serotypes 1, 6, 7, 8 and 9. In a final example, where the target tissue is the lungs, the AAV may be selected from serotypes 4, 5, 6 and 9.

In an alternative embodiment, the vector is a non-viral vector, such as a plasmid. In this embodiment, a non-viral vector may be delivered to a target cell or tissue using transfection. Examples of suitable transfection techniques would be known to the skilled person and include chemical and physical transfection. In one embodiment, chemical transfection includes the use of calcium phosphate, lipid or protein complexes. In one example, the non-viral vector may be combined with a lipid solution to result in the formation of a liposome or lipoplex. In another embodiment, physical transfection means include electroporation, microinjection or the use of ballistic particles. In a further example, bacteria can be used to deliver a non-viral vector to a target cell or tissue. This is known as bactofection.

In another aspect of the invention there is provided a host cell comprising the vector described above.

The “host cell” or “cell” as used herein may be eukaryotic, and may include bacterial cells, fungal cells such as yeast, plant cells, insect cells, or mammalian cells (human or non-human cells). In a preferred example, the host cell may be selected from a heart, brain, liver, eye, kidney, gut, pancreas, muscle or lung cell. In a more preferred embodiment, the cell may be selected from a heart, brain, muscle or lung cell. In one embodiment, the cell is a heart cell.

In another aspect of the invention there is provided a transgenic organism where the transgenic organism expresses a nucleic acid molecule of the invention. Again, the organism is any prokaryote or eukaryote.

In one embodiment, the progeny organism is transiently transformed with the nucleic acid molecule. In another embodiment, the progeny organism may be stably transformed with the nucleic acid molecule described herein and comprises the exogenous polynucleotide which is heritably maintained in at least one cell of the organism. The method may include steps to verify that the construct or vector is stably integrated.

In a further aspect of the invention, there is provided an organism obtained or obtainable by the methods described herein.

The term “organism” as used herein refers to any prokaryotic or eukaryotic organism. Some examples of eukaryotes include a human, a non-human primate/mammal, a livestock animal (e.g. cattle, horse, pig, sheep, goat, chicken, camel, donkey, cat, and dog), a mammalian model organism (mouse, rat, hamster, guinea pig, rabbit or other rodents), an amphibian (e.g., Xenopus), fish, insect (e.g. Drosophila), a nematode (e.g., C. elegans), a plant, an algae, a fungus. Examples of prokaryotes include bacteria (e.g. cyanobacteria) and archaea. In a most preferred embodiment, the organism is not a human.

In a further aspect of the invention there is provided a composition comprising the nucleic acid molecule described herein. Optionally the composition may further comprise a pharmaceutically acceptable carrier. Such pharmaceutically acceptable carriers may comprise excipients and other components which facilitate processing of the active compounds into preparations suitable for pharmaceutical administration.

In a further aspect of the invention there is provided a nucleic acid molecule or composition as described herein for use as a medicament. In another aspect of the invention, there is provided a method of therapy, the method comprising administering, preferably a therapeutically effective amount of the nucleic acid molecule or composition as described herein to an individual or patient in need thereof.

In another aspect of the invention, there is provided a nucleic acid molecule or composition as described above for use in the treatment of a mitochondrial disease. Alternatively, there is provided a method of treating a mitochondrial disease, the method comprising administering a nucleic acid molecule or composition as described above for use in the treatment of a mitochondrial disease.

As used herein a “mitochondrial disease” is a condition, disease, or disorder characterized by a defect in activity or function of mitochondria, particularly a defect in mitochondrial activity or function that results from, or is associated with, a mutation in mtDNA. Examples of mitochondrial disorders include, without limitation, ageing; AD (Alzheimer's Disease); ADPD (Alzeimer's Disease and Parkinsons's Disease); aminoglycoside-induced deafness; cancer; cardiomyopathy; CPEO (chronic progressive external ophthalmoplegia); encephalomyopathy; FBSN (familial bilateral striatal necrosis); FICP (Fatal Infantile Cardiomyopathy Plus, a MELAS-associated cardiomyopathy); LDYT (Leber's hereditary optic neuropathy and DysTonia); LHON (Leber hereditary optic neuropathy); LIMM (Lethal Infantile Mitochondrial Myopathy); MM (Mitochondrial Myopathy); MMC (Maternal Myopathy and Cardiomyopathy); MELAS (mitochondrial myopathy, encephalopathy, lactic acidosis, and stroke-like episodes): MERRF (myoclonic epilepsy with stroke-like episodes); MERRF (Myoclonic Epilepsy and Ragged Red Muscle Fibers); MILS (maternally-inherited Leigh syndrome); mitochondrial myopathy; NARP (Neurogenic muscle weakness, Ataxia, and Retinitis Pigmentosa; alternate phenotype at this locus is reported as Leigh Disease); PEO; SNE (subacute necrotizing encephalopathy); MHCM (Maternally inherited Hypertrophic CardioMyopathy); CPEO (Chronic Progressive External Ophthalmoplegia); KSS (Kearns Sayre Syndrome); DM (Diabetes Mellitus); DMDF (Diabetes Mellitus+DeaFness); CIPO (Chronic Intestinal Pseudoobstruction with myopathy and Ophthalmoplegia); DEAF (Maternally inherited DEAFness or aminoglycoside-induced DEAFness); PEM (Progressive encephalopathy) and SNHL (SensoriNeural Hearing Loss). In another embodiment, a mitochondrial disease is a disease associated with any mutation in mtDNA. For example, around 60% of all tumours contain mtDNA mutations, many of which result in levels of heteroplasmy, or mutation homoplasmy, that cause mitochondrial dysfunction.

A “therapeutically effective amount” as used herein may refer to an amount that is suitable to be therapeutically effective at the dosage and for the periods of time necessary to achieve the therapeutic purpose. The skilled person will appreciate that the amount to be administered will vary depending on such factors as the age, sex, weight of the individual. A therapeutically effective amount may also preferably be an amount that limits any unwanted side-effects on the treatment.

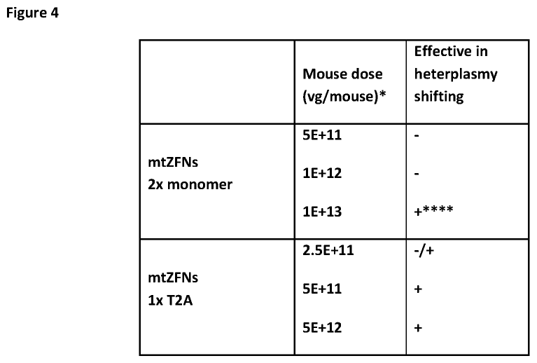

Of note the inventors have shown in FIGS. 2 and 4 that the dose needed to achieve a change in mitiochondrial DNA heteroplasmy is significantly lower when a nucleic acid molecule of the invention is administered compared to when two nucleic acid molecules are used to administer the same proteins. In particular, as can be seen from the table in FIG. 4, a significant shift in heteroplasmy is only observed at a concentration of 1×10¹³ vg/mouse. In comparison, a shift in heteroplasmy can be observed at a dose as low as 2.5×10¹¹ vg/mouse with the single nucleic acid molecule (the comparable dose of 5×10¹¹ vg/mouse for the 2 virions has no effect on heteroplasmy).

Accordingly, the therapeutically effective dose for administration of a nucleic acid molecule of the present invention will be lower than the therapeutically effective dose required for administration of the same proteins in two or more nucleic acid molecules.

Administration of the nucleic acid molecule may be accomplished by physical disturbance. Methods of physical disturbance include electroporation, gene guns, ultrasound or high-pressure injection.

Administration of the nucleic acid molecule or the composition may be accomplished orally or parenterally. Methods of parenteral delivery include topical, intra-arterial, intramuscular, subcutaneous, intramedullary, intrathecal, intraventricular, intravenous, intraperitoneal, mucosal or intranasal administration. Most preferably however the nucleic acid molecule or composition is administered by local or systemic injection. Local injection encompasses electroporation, gene gun, ultrasound and high pressure; whilst systemic injection encompasses vein injection, portal injection and artery injection. For example, where the target tissue is the heart, the nucleic acid molecule(s) may be administered directly into the heart concurrently with a surgical procedure for a stent or coronary artery bypass, for example.

Pharmaceutical compositions for oral administration can be formulated using pharmaceutically acceptable carriers known in the art in dosages suitable for oral administration. Such carriers enable the compositions to be formulated as tablets, pills, dragees, capsules, liquids, gels, syrups, slurries, suspensions, and the like suitable for ingestion by the subject.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous suspension injections can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilisers or agents which increase the solubility of the compounds to allow for the preparation of highly concentrated solutions. Pharmaceutical compositions may also include adjuvants to enhance or modulate antigenicity.

For topical or nasal administration, penetrants appropriate to the particular barrier to be permeated may be used in the formulation.

In another aspect of the invention, there is provided a method of changing mitochondrial DNA heteroplasmy the method comprising administering the nucleic acid molecule of the present invention to a target cell or target tissue. As explained already, there are thousands of copies of mtDNA in every cell. Within this population there exists a large number of mutations, some of which will be pathogenic mutations. The variable phenotypic expression of pathogenic mutations depends upon the degree of heteroplasmy and the energy requirements of the affected tissue. Accordingly, each tissue has a threshold concentration of mtDNA that must be exceeded to cause disease. This results in a heterogenous population of mtDNA comprising both wild-type and pathogenic mtDNA genomes. In a preferred embodiment of the invention, the method comprises changing or shifting the ratio of wild-type to pathogenic mtDNA—that is, increasing the amount of wild-type to pathogenic mtDNA. Preferably, the shift may be at least 5%, 10%, 15%, 20%, 25%, 30%, 35%, 40%, 45%, 50%, 55%, 60%, 65%, 70%, 75%, 80%, 85%, 90% or 99% in favour of the wild-type mtDNA genome. Preferably the shift in the wild-type: pathogenic ratio causes the concentration of pathogenic mtDNA to fall below the disease-causing threshold for that tissue.

In a further aspect of the invention, there is provided a method of introducing a single-strand and/or double-strand break into at least one mtDNA, preferably a pathogenic mtDNA, the method comprising administering the nucleic acid molecule of the present invention to a target cell or target tissue.

In a final aspect of the invention, there is provided a method for the simultaneous expression and delivery to the mitochondria of at least two proteins—a first and a second protein, as described above, the method comprising administering the nucleic acid molecule of the present invention to a target cell or target tissue. In a preferred embodiment, the expression and/or rate of import of the first protein is higher than the expression and/or rate of import of the second protein in the mitochondria. As explained above, and shown in FIG. 2, when the ribosomal skipping sequence is a 2A peptide or when the second protein comprises an additional amino acid, such as a proline, on its N-terminal MLS sequence, the MLS is masked resulting in a slower rate of import of the second protein into the mitochondria.

Again, the dose of the nucleic acid molecule of the present invention for use in the above methods will be lower than the dose required for administration of the same proteins in two or more nucleic acid molecules.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

As used herein, a control may be an individual, patient or cell that has not been treated with at least one nucleic acid molecule of the invention.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appin cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting example.

### EXAMPLE I

Testing the Expression and Heteroplasmy Shift by mtZFN Monomers Separated by 2A Peptides

We aimed to test if both mtZFN monomers can be expressed and specifically shift heteroplasmy in cultured cells if separated with a 2A peptide. The 2A approach relies upon skipping the synthesis of the glycyl-prolyl peptide bond at the C-terminus of 2A, and this sequence being placed within an ORF that encodes more than one protein of interest, and between these two proteins. This results in the upstream protein being released from the ribosome by eukaryotic release (termination) factors 1 and 3 (eRF1, eRF3). The released polypeptide retains the majority of the 2A peptide on its C-terminus. The ribosome then resumes translation of mRNA, with the downstream protein containing a proline on its N-terminus. The presence of a C-terminal extension in the upstream mtZFN monomer and a proline on the N-terminus of the downstream mtZFN monomer could in theory interfere with the nucleolytic activity of FokI domains (protein 1) and/or mitochondrial targeting of mtZFNs (protein 2). To test this experimentally, we performed heteroplasmy shifting experiments using the previously described mtZFN (COMP/NARPd) in cybrid cells harbouring the m.8993T>G mutation (FIG. 1). In contrast to the previous experiments, where the COMP and NARPd monomers were expressed from separate plasmids, they were now placed with in once vector and separated with DNA sequence coding for various types of 2A peptides (T2A, *T2A, *P2A) (FIGS. 1b and 1d). We detected substantial mtDNA heteroplasmy shift in all conditions as measured by RFLP of last-cycle hot PCR products (mtDNA nt positions 8339-9334) amplified from total DNA samples of cells harboring indicated levels of m.8993T>G (FIG. 1f). This result is consistent with effective separation of mtZFN monomers by the action of T2A and their effective mitochondrial import. The processing of the mitochondrial localisation signal (MLS) in each of mtZFN monomers has been detected by western blotting (FIG. 1e). This constitutes the first example of using 2A peptides for mitochondrial import of two ZFNs expressed from one bi-cistron under control of the same promoter. These results also show that the C-terminal extension on the upstream mtZFN monomer and the N-terminal proline on the downstream mtZFN monomer do not critically interfere with DNA nucleolysis by FokI and/or mitochondrial import, respectively. Importantly, the N-terminal proline appears to slow the rate of import of the second mtZFN (COMPa(−)) by masking the MLS. This enhances the efficiency of heteroplasmy shifting in mitochondria, as otherwise high expression levels of both monomers can lead to off-target effects (Gammage et al., 2016, Nucleic Acids Res.). This is the first time masking of a downstream MLS has been demonstrated using 2A peptides, which in this context allows critical regulation of the catalytic rate of mtZFN-mediated DNA cleavage in mitochondria.

Testing mtDNA Heteroplasmy Shift in Vivo by mtZFN Monomers Separated by 2A Peptides

We set out to determine if mtZFN monomers separated by the T2A peptide will be effective in targeting mtDNA in vivo. We selected T2A for this work as it is the shortest of the x2A peptides tested and works with comparable efficiency (FIG. 1). As a model we used the only available heteroplasmic mouse, which recapitulates key molecular features of mitochondrial disorders in cardiac tissue. This mouse strain bears the point mutation m.5024C>T in mitochondrial tRNA^{ALA} (mt-tRNA^{ALA}). We tested the mtZFN monomer pair (MTM25 and WTM1) previously showed to shift mtDNA heteroplasmy in vivo when comprised within two separate viral genomes and encapsidated within an AAV vector (Gammage et al., 2018, Nat. Med.). We encoded MTM25 and WTM1 mtZFN monomers in a single viral genome (FIG. 2a) and encapsidated within the same cardiac-tropic, engineered AAV9.45 serotype. Initial attempts to obtain AAV9.45 containing MTM25-T2A-WTM1 resulted in large-scale deletions within the encapsidated genomes. We reasoned that these deletions stem from recombination of the highly repetitive sequences of the tandemly arranged mtZFNs. To overcome this problem, we have recoded the mtZFN monomers, so that they share 63% sequence homology (i.e. sequence identity) rather than 93%, with no stretch of homology longer than 9 bp (previously >400). This recoded construct was then used again to generate the cardiac-tropic AAV9.45, resulting in high titres of viral particles with full length, faithfully encoded construct.

Next, various doses of MTM25-T2A-WTM-AAV9.45 (2.5*10¹¹, 5*10¹¹ and 5*10¹² vg/mouse) were administered into mt-tRNA^{ALA} animals harbouring m.5024C>T heteroplasmy ranging from ˜50%-80% (FIG. 2b). To allow a direct comparison between our previous disclosure (Gammage et al., 2018) and the current disclosure, these doses of MTM25-T2A-WTM-AAV9.45 are halved as compared to the separate monomer AAV doses to reflect the simultaneous delivery of both mtZFN monomers within every transduced cell. According to this: 5*10¹¹ separate 2x AAV monomer dose is an equivalent of 2.5*10¹¹ T2A dose. As only minimal variance in heteroplasmy is observed between tissues of the m.5024C>T mouse, mtDNA heteroplasmy is assessed by comparison of pyrosequencing data, expressed as the change (Δ) between ear punch genotype (E) determined at two weeks of age (prior to experimental intervention) and post-mortem heart genotype (H). Analysis of animals at 65 days post-injection revealed specific elimination of the m.5024C>T mutant mtDNA in mtZFN-treated mice, but not in vehicle controls (FIG. 2c). The extent to which heteroplasmy was altered by mtZFN treatment followed AAV dose-dependent trend, with a dose as low as 2.5*10¹¹ vg being partially effective and a dose of 5*10¹¹ proving efficient in elimination of m.5024C>T mutant mtDNA (FIG. 2c, outlined bar). This constitutes a great improvement as compared to our previous approach that relied on injecting MTM25 and WTM1 mtZFN monomers as separate viral particles. In these previous experiments the lowest effective dose was 5*10¹², whereas 5*10¹¹ and 1*10¹² were ineffective due to insufficient concentration of mtZFNs and mosaic transduction of the targeted tissue by AAV. The highest dose (1*10¹³ vg/mouse) used for MTM25 and WTM1 mtZFN monomers encapsidated in separate virions resulted in partial mtDNA copy number depletions due to off target effect, which are not observed in the equivalent dose of MTM25-T2A-WTM-AAV9.45 (FIG. 2d, striped bar). There were no detectable changes in mtDNA copy number in any other conditions.

### Materials and Methods

FIG. 1: A, B—FACS methods detailed in Gammage et al 2016, Methods Mol Biol; D-F—Cloning, western blotting and radio labelled PCR methods detailed Gammage et al., 2014 EMBO Mol Med.

FIG. 2: A—Synthesis of entire recoded construct from commercial supplier (GeneArt), Cloning as in Gammage et al., 2014 EMBO Mol Med. B—Cloning as described in Gammage et al., 2018 Nat Med and Gammage et al., 2014 EMBO Mol Med. C, D—Tissue extraction and DNA analysis as in Gammage et al., 2018 Nat Med.

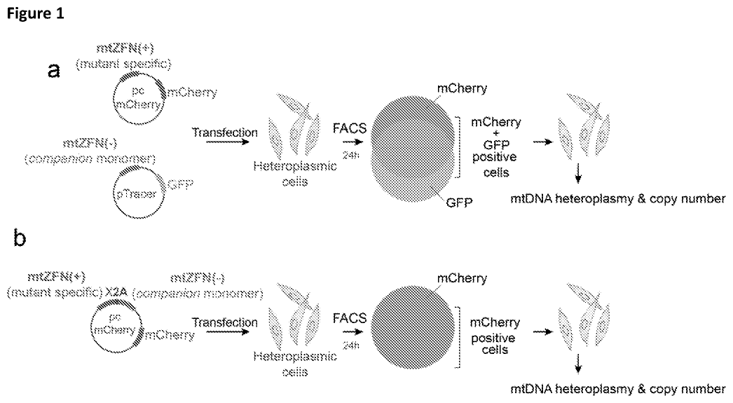

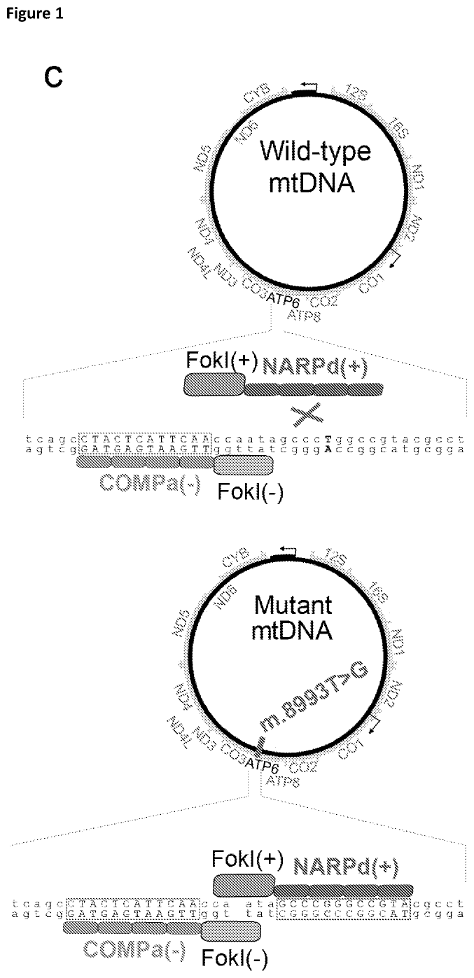

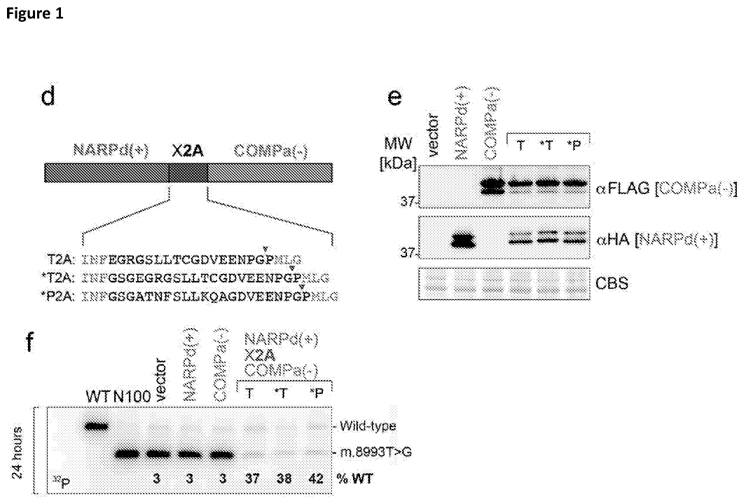

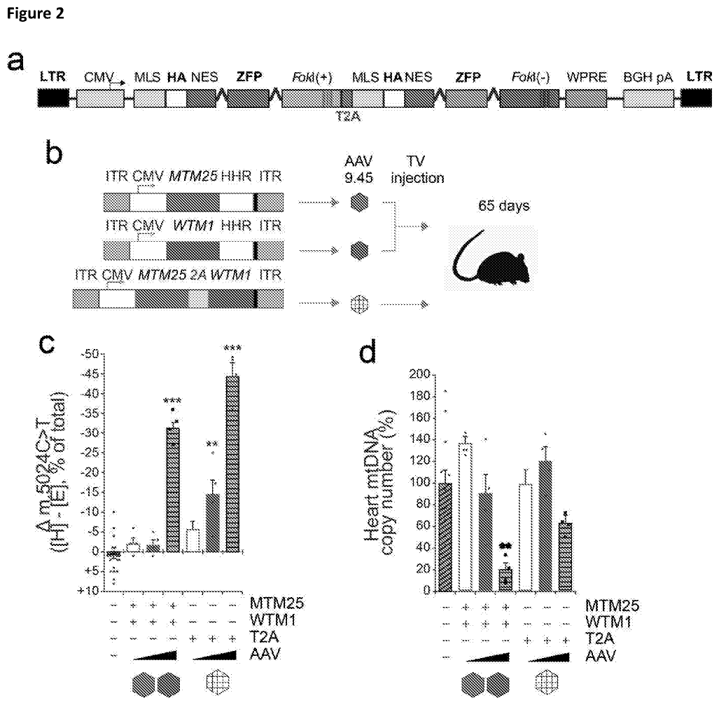

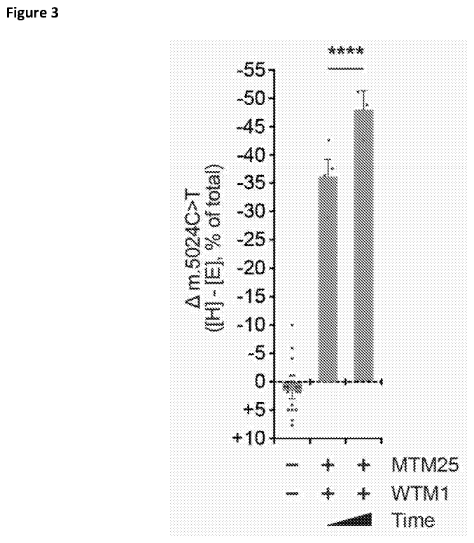

FIG. 3. Tissue extraction and DNA analysis as in Gammage et al., 2018 Nat Med.

### Description of the Evidence for Recombination During AAV Generation

Following encapsidation of the full-length construct into AAV9.45, at two separate commercial AAV preparation facilities, a PCR product spanning the length of the transgene from CMV promoter to BGH polyadenylation site was generated, demonstrating significantly diminished molecular weight (˜2 kb) when compared with a PCR product generated from the plasmid of origin (—3.5 kb). Upon Sanger sequencing of these PCR products, frameshifting recombination events were apparent between regions of significant homology in the AAV sample (between MLS sequences, ZFPs and FokI domains) that were not present in the plasmid. Upon recoding mtZFN2, making use of the redundancy in codon usage, a reduction in sequence identity between mtZFN1 and mtZFN2 at the nucleotide level was achieved, going from 92% to 63%. In addition, the longest stretch of homologous sequence between mtZFN1 and mtZFN2 was reduced from 424 bp to 9 bp. Following encapsidation of the full-length recoded construct, no differences in molecular weight between plasmid or AAV derived PCR products spanning the transgene region could be detected, and no recombined molecules were detected by Sanger sequencing.

### REFERENCES

Minczuk M et al., 2006. Sequence-specific modification of mitochondrial DNA using a chimeric zinc finger methylase. Proc. Natl. Acad. Sci. USA. 103(52): 19689-19694.

Minczuk M et al., 2010. Construction and testing of engineered zinc-finger proteins for sequence-specific modification of mtDNA. Nat. Protoc. 5(2): 342-356.

Gammage et al., 2014. Mitochondrially targeted ZFNs for selective degradation of pathogenic mitochondrial genomes bearing large-scale deletions or point mutations. EMBO Mol. Med. 6(4): 458-466.

Gammage et al., 2016. Near-complete elimination of mutant mtDNA by iterative or dynamic dose-controlled treatment with mtZFNs. Nucleic Acids Res. 44(16): 7804-7816.

Gammage et al., 2018. Genome editing in mitochondria corrects a pathogenic mtDNA mutation in vivo. Nat. Med. Doi: 10.1038/s41591-018-0165-9.

Cermak, T et al., Efficient design and assembly of custom TALEN and other TAL effector-based constructs for DNA targeting. Nucleic acid Res. 39 (2011).

SEQUENCE LISTING
Amino acid sequences:
SEQ ID NO: 1: Mitochondrial localisation signal 1
MLGFVGRVAAAPASGALRRLTPSASLPPAQLLLRAAPTAVHPVRDYAAQ
SEQ ID NO: 2: Mitochondrial localisation signal 2
PMLGFVGRVAAAPASGALRRLTPSASLPPAQLLLRAAPTAVHPVRDYAAQ
SEQ ID NO: 3: HA epitope tag
YPYDVPDYA
SEQ ID NO: 4: Nuclear export tag
VDEMTKKFGTLTIHDTEK
SEQ ID NO: 5: Short flexible linker
AA
SEQ ID NO: 6: Restriction site
EF
SEQ ID NO: 7: Zn Finger protein 1
MAERPFQCRICMRNFSGNTGLNCHIRTHTGEKPFACDICGRKFADRSNLTRHTKIHTH
PRAPIPKPFQCRICMRNFSQSGSLTRHIRTHTGEKPFACDICGRKFAHKSARAAHTKIH
TGSQKPFQCRICMRNFSRSDHLSAHIRTHTGEKPFACDICGRKFAQHGSLASHTKIHL
R
SEQ ID NO: 8: Fokl catalytic domain 1
QLVKSELEEKKSELRHKLKYVPHEYIELIEIARNSTQDRILE
MKVMEFFMKVYGYRGKHLGGSRKPDGAIYTVGSPIDYGVIVDTKAYSGGYNLPIGQA
DEMQRYVKENQTRNKHINPNEWWKVYPSSVTEFKFLFVSGHFKGNYKAQLTRLNRK
TNCNGAVLSVEELLIGGEMIKAGTLTLEEVRRKFNNGEINF
SEQ ID NO: 9: Zn Finger protein 2
MAERPFQCRICMRNFSLPHHLEQHIRTHTGEKPFACDICGRKFARN
ASRTRHTKIHTGSQKPFQCRICMRKFAYTYSLSEHTKIHTGEKPFQCRICMRNFSQSA
NRTTHIRTHTGEKPFACDICGRKFAHRSSLRRHTKIHLR
SEQ ID NO: 10: Fokl catalytic domain 2
QLVKSELEEKKSELRHKLKYVPHEYIELIEIARNSTQDRILEMKVMEFFMKVYGYRGKH
LGGSRKPDGAIYTVGSPIDYGVIVDTKAYSGGYNLPIGQADEMERYVEENQTRDKHLN
PNEWWKVYPSSVTEFKFLFVSGHFKGNYKAQLTRLNHITNCNGAVLSVEELLIGGEMI
KAGTLTLEEVRRKFNNGEINF
SEQ ID NO: 11: T2A sequence
EGRGSLLTCGDVEENPG
Nucleic acid sequences
SEQ ID NO: 12: Mitochondrial localisation signal 1
ATGTTGGGGTTTGTGGGTCGGGTGGCCGCTGCTCCGGCCTCCGGGGCCTTGCG
GAGACTCACCCCTTCAGCGTCGCTGCCCCCAGCTCAGCTCTTACTGCGGGCCGC
TCCGACGGCGGTCCATCCTGTCAGGGACTATGCGGCGCAA
SEQ ID NO: 13: Mitochondrial localisation signal 2
ATGCTCGGTTTCGTAGGCCGCGTCGCTGCCGCACCAGCTTCAGGTGCACTCCGC
CGATTGACACCCAGCGCAAGCCTTCCTCCCGCACAGTTGTTGCTCCGAGCTGCC
CCCACCGCCGTTCACCCCGTGCGAGATTACGCAGCTCAG
SEQ ID NO: 14: HA epitope tag
TACCCCTACGACGTGCCCGACTACGCC
SEQ ID NO: 15: Nuclear export tag
GTGGATGAAATGACCAAAAAGTTCGGCACGCTCACCATTCACGACACCGAAAAG
SEQ ID NO: 16: Short flexible linker
GCCGCC
SEQ ID NO: 17: Restriction site
GAATTC
SEQ ID NO: 18: Zn Finger protein 1
ATGGCTGAGAGGCCCTTCCAGTGTCGAATCTGCATGCGTAACTTCAGTGGCAACA
CCGGCCTGAACTGTCACAICCGCACCCACACCGGCGAGAAGCCTTTTGCCTGTG
ACATTTGTGGGAGGAAATTTGCCGACCGCTCCAACCTGACCCGCCATACCAAGAT
ACACACGCATCCCAGGGCACCTATTCCCAAGCCCTTCCAGTGTCGAATCTGCATG
CGTAACTTCAGTCAGTCCGGCTCCCTGACCCGCCACATCCGCACCCACACCGGC
GAGAAGCCTTTTGCCTGTGACATTTGTGGGAGGAAATTTGCCCACAAGTCCGCCC
GCGCCGCCCATACCAAGATACACACGGGATCTCAGAAGCCCTTCCAGTGTCGAAT
CTGCATGCGTAACTTCAGTCGCTCCGACCACCTGTCCGCCCACATCCGCACCCAC
ACCGGCGAGAAGCCTTTTGCCTGTGACATTTGTGGAGGAAATTTGCCCAGCACG
GCTCCCTGGCCTCCCATACCAAGATACACCTGCGG
SEQ ID NO: 19: Fokl catalytic domain 1
CAGCTGGTGAAGAGCGAGCTGGAGGAGAAGAAGTCCGAGCTGCGGCACAAGCT
GAAGTACGTGCCCCACGAGTACATCGAGCTGATCGAGATCGCCAGGAACAGCAC
CCAGGACCGCATCCTGGAGATGAAGGTGATGGAGTTCTTCATGAAGGTGTACGG
CTACAGGGGAAAGCACCTGGGCGGAAGCAGAAAGCCTGACGGCGCCATCTATAC
AGTGGGCAGCCCCATCGATTACGGCGTGATCGTGGACACAAAGGCCTACAGCGG
CGGCTACAATCTGCCTATCGGCCAGGCCGACGAGATGCAGAGATACGTGAAGGA
GAACCAGACCCGGAATAAGCACATCAACCCCAACGAGTGGTGGAAGGTGTACCC
TAGCAGCGTGACCGAGTTCAAGTTCCTGTTCGTGAGCGGCCACTTCAAGGGCAAC
TACAAGGCCCAGCTGACCAGGCTGAACCGCAAGACCAACTGCAATGGCGCCGTG
CTGAGCGTGGAGGAGCTGCTGATCGGCGGCGAGATGATCAAAGCCGGCACCCT
GACACTGGAGGAGGTGCGGCGCAAGTTCAACAACGGCGAGATCAACTTC
SEQ ID NO: 20: Zn Finger protein 2
ATGGCCGAACGCCCTTTTCAATGCCGGATTTGTATGAGAAATTTTTCTCTTCCTCA
CCACCTGGAGCAACATATTAGGACACATACTGGGGAAAAACCCTTCGCTTGCGAT
ATCTGCGGACGCAAGTTCGCTCGGAATGCTTCCCGCACTCGACACACAAAAATCC
ATACAGGGTCCCAGAAGCCATTCCAATGCAGGATCTGCATGAGAAAGTTCGCATA
CACCTACTCTCTCTCTGAGCATACTAAAATTCACACTGGGGAAAAACCATTTCAAT
GCAGAATATGTATGCGAAATTTCTCCCAGAGTGCTAATCGGACCACTCATATTCGA
ACACATACAGGAGAAAAACCCTTCGCTTGCGATATCTGCGGACGAAAGTTCGCTC
ATAGGAGTAGCCTCCGCCGCCACACAAAAATCCATCTTCGC
SEQ ID NO: 21: Fokl catalytic domain 2
CAACTTGTTAAATCAGAACTCGAAGAAAAAAAAAGCGAGCTACGCCATAAACTCAA
ATATGTACCTCATGAATATATTGAATTAATTGAAATTGCAAGAAATAGTACACAAGA
TCGAATTTTGGAAATGAAAGTCATGGAATTTTTTATGAAAGTATATGGTIACCGCG
GCAAACATCTTGGAGGATCAAGGAAACCAGATGGGGCAATTTACACTGTTGGGAG
TCCTATAGACTACGGGGTCATTGTCGATACCAAAGCTTATTCTGGAGGGTATAACC
TTCCCATTGGTCAAGCTGATGAAATGGAGCGCTATGTAGAAGAAAATCAAACAAGA
GACAAACATCTTAACCCTAATGAATGGTGGAAAGTCTATCCCAGTTCTGTTACTGA
ATTTAAATTCTCTTTGTCTCTGGACATTTTAAAGGAAATTATAAAGCTCAACTCAO
AAGATTAAATCATATAACAAATTGTAACGGTGCTGTACTCTCAGTCGAAGAACTCC
TCATTGGAGGTGAAATGATAAAGGCTGGAACACTCACCCTCGAAGAAGTTCGCCG
AAAATTAATTAATGGGGAAATTAATTTT
SEQ ID NO: 22: T2A sequence
GAAGGGAGAGGATCTCTGCTTACTTGCGGCGATGTAGAGGAAAACCCCGGACCC
SEQ ID NO: 23: Restriction site
GS
SEQ ID NO: 24: Restriction site
GGATCC
SEQ ID NO: 25: Complete MTM25(+)_T2A_WTM1(−) amino acid
sequence
MLGFVGRVAAAPASGALRRLTPSASLPPAQLLLRAAPTAVHPVRDYAAQYPYDVPDY
AVDEMTKKFGTLTIHDTEKAAEFMAERPFQCRICMRNFSGNTGLNCHIRTHTGEKPFA
CDICGRKFADRSNLTRHTKIHTHPRAPIPKPFQCRICMRNFSQSGSLTRHIRTHTGEKP
FACDICGRKFAHKSARAAHTKIHTGSQKPFQCRICMRNFSRSDHLSAHIRTHTGEKPF
ACDICGRKFAQHGSLASHTKIHLRGSQLVKSELEEKKSELRHKLKYVPHEYIELIEIARN
STQDRILEMKVMEFFMKVYGYRGKHLGGSRKPDGAIYTVGSPIDYGVIVDTKAYSGG
YNLPIGQADEMQRYVKENQTRNKHINPNEWWKVYPSSVTEFKFLFVSGHFKGNYKA
QLTRLNRKTNCNGAVLSVEELLIGGEMIKAGTLTLEEVRRKFNNGEINFLEEGRGSLLT
CGDVEENPGPMLGFVGRVAAAPASGALRRLTPSASLPPAQLLLRAAPTAVHPVRDYA
AQYPYDVPDYAVDEMTKKFGTLTIHDTEKAAEFMAERPFQCRICMRNFSLPHHLEQHI
RTHTGEKPFACDICGRKFARNASRTRHTKIHTGSQKPFQCRICMRKFAYTYSLSEHTK
IHTGEKPFQCRICMRNFSQSANRTTHIRTHTGEKPFACDICGRKFAHRSSLRRHTKIHL
RGSQLVKSELEEKKSELRHKLKYVPHEYIELIEIARNSTQDRILEMKVMEFFMKVYGYR
GKHLGGSRKPDGAIYTVGSPIDYGVIVDTKAYSGGYNLPIGQADEMERYVEENQTRD
KHLNPNEWWKVYPSSVTEFKFLFVSGHFKGNYKAQLTRLNHITNCNGAVLSVEELLIG
GEMIKAGTLTLEEVRRKFNNGEINF
SEQ ID NO: 26: Complete MTM25(+)_T2A_WTM1(−) nucleic amino
acid sequence
ATGTTGGGGTTTGTGGGTCGGGTGGCCGCTGCTCCGGCCTCCGGGGCCTTGCG
GAGACTCACCCCTTCAGCGTCGCTGCCCCCAGCTCAGCTCTTACTGCGGGCCGC
TCCGACGGCGGTCCATCCTGTCAGGGACTATGCGGCGCAATACCCCTACGACGT
GCCCGACTACGCCGTGGATGAAATGACCAAAAAGTTCGGCACGCTCACCATTCAC
GACACCGAAAAGGCCGCCGAATTCATGGCTGAGAGGCCCTTCCAGTGTCGAATC
TGCATGCGTAACTTCAGTGGCAACACCGGCCTGAACTGTCACATCCGCACCCACA
CCGGCGAGAAGCCTTTTGCCTGTGACATTTGTGGGAGGAAATTTGCCGACCGCTC
CAACCTGACCCGCCATACCAAGATACACACGCATCCCAGGGCACCTATTCCCAAG
CCCTTCCAGTGTCGAATCTGCATGCGTAACTTCAGTCAGTCCGGCTCCCTGACCC
GCCACATCCGCACCCACACCGGCGAGAAGCCTTTTGCCTGTGACATTTGTGGGA
GGAAATTTGCCCACAAGTCCGCCCGCGCCGCCCATACCAAGATACACACGGGAT
CTCAGAAGCCCTTCCAGTGTCGAATCTGCATGCGTAACTTCAGTCGCTCCGACCA
CCTGTCCGCCCACATCCGCACCCACACCGGCGAGAAGCCTTTTGCCTGTGACATT
TGTGGGAGGAAATTTGCCCAGCACGGCTCCCTGGCCTCCCATACCAAGATACACC
TGCGGGGATCCCAGCTGGTGAAGAGCGAGCTGGAGGAGAAGAAGTCCGAGCTG
CGGCACAAGCTGAAGTACGTGCCCCACGAGTACATCGAGCTGATCGAGATCGCC
AGGAACAGCACCCAGGACCGCATCCTGGAGATGAAGGTGATGGAGTTCTTCATG
AAGGTGTACGGCTACAGGGGAAAGCACCTGGGCGGAAGCAGAAAGCCTGACGG
CGCCATCTATACAGTGGGCAGCCCCATCGATTACGGCGTGATCGTGGACACAAA
GGCCTACAGCGGCGGCTACAATCTGCCTATCGGCCAGGCCGACGAGATGCAGAG
ATACGTGAAGGAGAACCAGACCCGGAATAAGCACATCAACCCCAACGAGTGGTG
GAAGGTGTACCCTAGCAGCGTGACCGAGTTCAAGTTCCTGTTCGTGAGCGGCCA
CTTCAAGGGCAACTACAAGGCCCAGCTGACCAGGCTGAACCGCAAGACCAACTG
CAATGGCGCCGTGCTGAGCGTGGAGGAGCTGCTGATCGGCGGCGAGATGATCAA
AGCCGGCACCCTGACACTGGAGGAGGTGCGGCGCAAGTTCAACAACGGCGAGAT
CAACTTCCTCGAGGAAGGGAGAGGATCTCTGCTTACTTGCGGCGATGTAGAGGAA
AACCCCGGACCCATGCTCGGTTTCGTAGGCCGCGTCGCTGCCGCACCAGCTTCA
GGTGCACTCCGCCGATTGACACCCAGCGCAAGCCTTCCTCCCGCACAGTTGTTG
CTCCGAGCTGCCCCCACCGCCGTTCACCCCGTGCGAGATTACGCAGCTCAGTAT
CCTTATGATGTCCCTGATTATGCTGTAGACGAGATGACAAAGAAATTTGGAACTTT
GACAATACATGATACAGAGAAAGCTGCAGAATTCATGGCCGAACGCCCTTTTCAAT
GCCGGATTTGTATGAGAAATTTTTCTCTTCCTCACCACCTGGAGCAACATATTAGG
ACACATACTGGGGAAAAACCCTTCGCTTGCGATATCTGCGGACGCAAGTTCGCTC
GGAATGCTTCCCGCACTCGACACACAAAAATCCATACAGGGTCCCAGAAGCCATT
CCAATGCAGGATCTGCATGAGAAAGTTCGCATACACCTACTCTCTCTCTGAGCATA
CTAAAATTCACACTGGGGAAAAACCATTTCAATGCAGAATATGTATGCGAAATTTC
TCCCAGAGTGCTAATCGGACCACTCATATTCGAACACATACAGGAGAAAAACCCTT
CGCTTGCGATATCTGCGGACGAAAGTTCGCTCATAGGAGTAGCCTCCGCCGCCA
CACAAAAATCCATCTTCGCGGATCCCAACTTGTTAAATCAGAACTCGAAGAAAAAA
AAAGCGAGCTACGCCATAAACTCAAATATGTACCTCATGAATATATTGAATTAATTG
AAATTGCAAGAAATAGTACACAAGATCGAATTTTGGAAATGAAAGTCATGGAATTTT
TTATGAAAGTATATGGTTACCGCGGCAAACATCTTGGAGGATCAAGGAAACCAGA
TGGGGCAATTTACACTGTTGGGAGTCCTATAGACTACGGGGTCATTGTCGATACC
AAAGCTTATTCTGGAGGGTATAACCTTCCCATTGGTCAAGCTGATGAAATGGAGC
GCTATGTAGAAGAAAATCAAACAAGAGACAAACATCTTAACCCTAATGAATGGTGG
AAAGTCTATCCCAGTCTGTTACTGAATTTAAATTCTCTTTGTCTCTGGACATTTTA
AAGGAAATTATAAAGCTCAACTCACAAGATTAAATCATATAACAAATTGTAACGGTG
CTGTACTCTCAGTCGAAGAACTCCTCATTGGAGGTGAAATGATAAAGGCTGGAAC
ACTCACCCTCGAAGAAGTTCGCCGAAAATTTAATAATGGGGAAATTAATTTT

1. A nucleic acid molecule for simultaneous expression and delivery to the mitochondria of at least two proteins, the nucleic acid molecule comprising a first nucleic acid sequence encoding a first mitochondrial localisation signal and a first protein and a second nucleic acid sequence encoding a second mitochondrial localisation signal and a second protein, wherein the first and second nucleic acid sequence are separated by at least one ribosomal skipping sequence and wherein the first and second nucleic acid sequences are operably linked to a regulatory sequence.

2. The nucleic acid molecule of claim 1, wherein the first and second nucleic acid sequences encode a first and second protein, wherein the percent sequence identity of the amino acid sequences is higher than the percent sequence identity of the nucleic acid sequences between the first and second protein.

3. The nucleic acid molecule of claim 2, wherein the first and second nucleic acid sequences encode proteins with a minimum of 70 to 90% amino acid sequence identity and a maximum of 55 to 70% nucleic acid sequence identity.

4. The nucleic acid molecule of claim 1, wherein the nucleic acid sequence of the first and second proteins do not have a stretch of sequence identity longer than 6 to 30 bp, more preferably 6 to 15 bp.

5. (canceled)

6. The nucleic acid molecule of claim 1, wherein the second mitochondrial localisation signal comprises one or more additional N-terminal amino acids that mask the mitochondrial localisation signal.

7. The nucleic acid molecule of claim 6, wherein the second mitochondrial localisation signal comprises an additional N-terminal proline.

8. The nucleic acid molecule of claim 1 wherein the first and second proteins both comprise a DNA-binding polypeptide and nuclease.

9. The nucleic acid molecule of claim 1, wherein the ribosomal skipping sequence is a nucleic acid sequence encoding a 2A peptide.

10. The nucleic acid molecule of claim 9, wherein the 2A peptide is selected from the group consisting of T2A, P2A, E2A and F2A.

11. The nucleic acid molecule of claim 8, wherein the DNA-binding polypeptide is a zinc finger DNA binding domain.

12. The nucleic acid molecule of claim 8, wherein the nuclease is FokI.

13. The nucleic acid molecule of claim 1, wherein the first and second nucleic sequences further encode a nuclear export signal.

14. The nucleic acid molecule of claim 1, wherein the nucleic acid molecule is contained within a vector.

15. The nucleic acid molecule of claim 14, wherein the vector is a viral or non-viral vector, preferably an adeno-associated virus.

16. The nucleic acid molecule of claim 1, wherein the regulatory sequence is a promoter.

17. A host cell comprising a nucleic acid molecule of claim 1.

18-19. (canceled)

20. A method of therapy, the method comprising administering the nucleic acid molecule of claim 1 to a patient or individual in need thereof.

21. A method of
(a) treating a mitochondrial disease;
(b) changing mitochondrial DNA heteroplasmy;
(c) introducing a single-strand and/or double-strand break into at least one mitochondrial DNA; or,
(d) simultaneous expression and delivery to the mitochondria of at least two proteins;
the method comprising administering the nucleic acid molecule of claim 1 to a patient or individual in need thereof, or a target cell or tissue.

22-24. (canceled)

25. The method of claim 21, wherein the expression and/or import of the first protein in the mitochondria is higher than the expression and/or import of the second protein in the mitochondria.


SEQUENCE LISTING (42 sequences)

SEQ ID NO: 1 - protein, 49 aa, Artificial Sequence
  Mitochondrial localisation signal 1
MLGFVGRVAAAPASGALRRLTPSASLPPAQLLLRAAPTAVHPVRDYAAQ

SEQ ID NO: 2 - protein, 50 aa, Artificial Sequence
  Mitochondrial localisation signal 2
PMLGFVGRVAAAPASGALRRLTPSASLPPAQLLLRAAPTAVHPVRDYAAQ

SEQ ID NO: 3 - protein, 9 aa, Artificial Sequence
  HA epitope tag
YPYDVPDYA

SEQ ID NO: 4 - protein, 18 aa, Artificial Sequence
  Nuclear export tag
VDEMTKKFGTLTIHDTEK

SEQ ID NO: 5-6 - skipped (2 entries)

SEQ ID NO: 7 - protein, 176 aa, Artificial Sequence
  Zn Finger protein 1
MAERPFQCRICMRNFSGNTGLNCHIRTHTGEKPFACDICGRKFADRSNLTRHTKIHTHPRAPIPKPFQCRICMRNFSQSGSLTRHIRTHTGEKPFACDICGRKFAHKSARAAHTKIHTGSQKPFQCRICMRNFSRSDHLSAHIRTHTGEKPFACDICGRKFAQHGSLASHTKIHLR

SEQ ID NO: 8 - protein, 196 aa, Artificial Sequence
  FokI catalytic domain 1
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 9 - protein, 143 aa, Artificial Sequence
  Zn Finger protein 2
MAERPFQCRICMRNFSLPHHLEQHIRTHTGEKPFACDICGRKFARNASRTRHTKIHTGSQKPFQCRICMRKFAYTYSLSEHTKIHTGEKPFQCRICMRNFSQSANRTTHIRTHTGEKPFACDICGRKFAHRSSLRRHTKIHLR

SEQ ID NO: 10 - protein, 196 aa, Artificial Sequence
  FokI catalytic domain 2
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 11 - protein, 17 aa, Artificial Sequence
  T2A sequence
EGRGSLLTCGDVEENPG

SEQ ID NO: 12 - DNA, 147 bp, Artificial Sequence
  Mitochondrial localisation signal 1
atgttggggtttgtgggtcgggtggccgctgctccggcctccggggccttgcggagactcaccccttcagcgtcgctgcccccagctcagctcttactgcgggccgctccgacggcggtccatcctgtcagggactatgcggcgcaa

SEQ ID NO: 13 - DNA, 147 bp, Artificial Sequence
  Mitochondrial localisation signal 2
atgctcggtttcgtaggccgcgtcgctgccgcaccagcttcaggtgcactccgccgattgacacccagcgcaagccttcctcccgcacagttgttgctccgagctgcccccaccgccgttcaccccgtgcgagattacgcagctcag

SEQ ID NO: 14 - DNA, 27 bp, Artificial Sequence
  HA epitope tag
tacccctacgacgtgcccgactacgcc

SEQ ID NO: 15 - DNA, 54 bp, Artificial Sequence
  Nuclear export tag
gtggatgaaatgaccaaaaagttcggcacgctcaccattcacgacaccgaaaag

SEQ ID NO: 16-17 - skipped (2 entries)

SEQ ID NO: 18 - DNA, 528 bp, Artificial Sequence
  Zn Finger protein 1
atggctgagaggcccttccagtgtcgaatctgcatgcgtaacttcagtggcaacaccggcctgaactgtcacatccgcacccacaccggcgagaagccttttgcctgtgacatttgtgggaggaaatttgccgaccgctccaacctgacccgccataccaagatacacacgcatcccagggcacctattcccaagcccttccagtgtcgaatctgcatgcgtaacttcagtcagtccggctccctgacccgccacatccgcacccacaccggcgagaagccttttgcctgtgacatttgtgggaggaaatttgcccacaagtccgcccgcgccgcccataccaagatacacacgggatctcagaagcccttccagtgtcgaatctgcatgcgtaacttcagtcgctccgaccacctgtccgcccacatccgcacccacaccggcgagaagccttttgcctgtgacatttgtgggaggaaatttgcccagcacggctccctggcctcccataccaagatacacctgcgg

SEQ ID NO: 19 - DNA, 588 bp, Artificial Sequence
  FokI catalytic domain 1
cagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaagaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 20 - DNA, 429 bp, Artificial Sequence
  Zn Finger protein 2
atggccgaacgcccttttcaatgccggatttgtatgagaaatttttctcttcctcaccacctggagcaacatattaggacacatactggggaaaaacccttcgcttgcgatatctgcggacgcaagttcgctcggaatgcttcccgcactcgacacacaaaaatccatacagggtcccagaagccattccaatgcaggatctgcatgagaaagttcgcatacacctactctctctctgagcatactaaaattcacactggggaaaaaccatttcaatgcagaatatgtatgcgaaatttctcccagagtgctaatcggaccactcatattcgaacacatacaggagaaaaacccttcgcttgcgatatctgcggacgaaagttcgctcataggagtagcctccgccgccacacaaaaatccatcttcgc

SEQ ID NO: 21 - DNA, 588 bp, Artificial Sequence
  FokI catalytic domain 2
caacttgttaaatcagaactcgaagaaaaaaaaagcgagctacgccataaactcaaatatgtacctcatgaatatattgaattaattgaaattgcaagaaatagtacacaagatcgaattttggaaatgaaagtcatggaattttttatgaaagtatatggttaccgcggcaaacatcttggaggatcaaggaaaccagatggggcaatttacactgttgggagtcctatagactacggggtcattgtcgataccaaagcttattctggagggtataaccttcccattggtcaagctgatgaaatggagcgctatgtagaagaaaatcaaacaagagacaaacatcttaaccctaatgaatggtggaaagtctatcccagttctgttactgaatttaaatttctctttgtctctggacattttaaaggaaattataaagctcaactcacaagattaaatcatataacaaattgtaacggtgctgtactctcagtcgaagaactcctcattggaggtgaaatgataaaggctggaacactcaccctcgaagaagttcgccgaaaatttaataatggggaaattaatttt

SEQ ID NO: 22 - DNA, 54 bp, Artificial Sequence
  T2A sequence
gaagggagaggatctctgcttacttgcggcgatgtagaggaaaaccccggaccc

SEQ ID NO: 23-24 - skipped (2 entries)

SEQ ID NO: 25 - protein, 895 aa, Artificial Sequence
  Complete MTM25(+)_T2A_WTM1(-) amino acid sequence
MLGFVGRVAAAPASGALRRLTPSASLPPAQLLLRAAPTAVHPVRDYAAQYPYDVPDYAVDEMTKKFGTLTIHDTEKAAEFMAERPFQCRICMRNFSGNTGLNCHIRTHTGEKPFACDICGRKFADRSNLTRHTKIHTHPRAPIPKPFQCRICMRNFSQSGSLTRHIRTHTGEKPFACDICGRKFAHKSARAAHTKIHTGSQKPFQCRICMRNFSRSDHLSAHIRTHTGEKPFACDICGRKFAQHGSLASHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFLEEGRGSLLTCGDVEENPGPMLGFVGRVAAAPASGALRRLTPSASLPPAQLLLRAAPTAVHPVRDYAAQYPYDVPDYAVDEMTKKFGTLTIHDTEKAAEFMAERPFQCRICMRNFSLPHHLEQHIRTHTGEKPFACDICGRKFARNASRTRHTKIHTGSQKPFQCRICMRKFAYTYSLSEHTKIHTGEKPFQCRICMRNFSQSANRTTHIRTHTGEKPFACDICGRKFAHRSSLRRHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 26 - DNA, 2685 bp, Artificial Sequence
  Complete MTM25(+)_T2A_WTM1(-) nucleic amino acid sequence
atgttggggtttgtgggtcgggtggccgctgctccggcctccggggccttgcggagactcaccccttcagcgtcgctgcccccagctcagctcttactgcgggccgctccgacggcggtccatcctgtcagggactatgcggcgcaatacccctacgacgtgcccgactacgccgtggatgaaatgaccaaaaagttcggcacgctcaccattcacgacaccgaaaaggccgccgaattcatggctgagaggcccttccagtgtcgaatctgcatgcgtaacttcagtggcaacaccggcctgaactgtcacatccgcacccacaccggcgagaagccttttgcctgtgacatttgtgggaggaaatttgccgaccgctccaacctgacccgccataccaagatacacacgcatcccagggcacctattcccaagcccttccagtgtcgaatctgcatgcgtaacttcagtcagtccggctccctgacccgccacatccgcacccacaccggcgagaagccttttgcctgtgacatttgtgggaggaaatttgcccacaagtccgcccgcgccgcccataccaagatacacacgggatctcagaagcccttccagtgtcgaatctgcatgcgtaacttcagtcgctccgaccacctgtccgcccacatccgcacccacaccggcgagaagccttttgcctgtgacatttgtgggaggaaatttgcccagcacggctccctggcctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaagaccaactgcaatggcgccgtgctgagcgtggaggagctgctgatcggcggcgagatgatcaaagccggcaccctgacactggaggaggtgcggcgcaagttcaacaacggcgagatcaacttcctcgaggaagggagaggatctctgcttacttgcggcgatgtagaggaaaaccccggacccatgctcggtttcgtaggccgcgtcgctgccgcaccagcttcaggtgcactccgccgattgacacccagcgcaagccttcctcccgcacagttgttgctccgagctgcccccaccgccgttcaccccgtgcgagattacgcagctcagtatccttatgatgtccctgattatgctgtagacgagatgacaaagaaatttggaactttgacaatacatgatacagagaaagctgcagaattcatggccgaacgcccttttcaatgccggatttgtatgagaaatttttctcttcctcaccacctggagcaacatattaggacacatactggggaaaaacccttcgcttgcgatatctgcggacgcaagttcgctcggaatgcttcccgcactcgacacacaaaaatccatacagggtcccagaagccattccaatgcaggatctgcatgagaaagttcgcatacacctactctctctctgagcatactaaaattcacactggggaaaaaccatttcaatgcagaatatgtatgcgaaatttctcccagagtgctaatcggaccactcatattcgaacacatacaggagaaaaacccttcgcttgcgatatctgcggacgaaagttcgctcataggagtagcctccgccgccacacaaaaatccatcttcgcggatcccaacttgttaaatcagaactcgaagaaaaaaaaagcgagctacgccataaactcaaatatgtacctcatgaatatattgaattaattgaaattgcaagaaatagtacacaagatcgaattttggaaatgaaagtcatggaattttttatgaaagtatatggttaccgcggcaaacatcttggaggatcaaggaaaccagatggggcaatttacactgttgggagtcctatagactacggggtcattgtcgataccaaagcttattctggagggtataaccttcccattggtcaagctgatgaaatggagcgctatgtagaagaaaatcaaacaagagacaaacatcttaaccctaatgaatggtggaaagtctatcccagttctgttactgaatttaaatttctctttgtctctggacattttaaaggaaattataaagctcaactcacaagattaaatcatataacaaattgtaacggtgctgtactctcagtcgaagaactcctcattggaggtgaaatgataaaggctggaacactcaccctcgaagaagttcgccgaaaatttaataatggggaaattaatttt

SEQ ID NO: 27 - protein, 18 aa, Artificial Sequence
  T2A
EGRGSLLTCGDVEENPGP

SEQ ID NO: 28 - protein, 21 aa, Artificial Sequence
  T2A*
GSGEGRGSLLTCGDVEENPGP

SEQ ID NO: 29 - protein, 20 aa, Artificial Sequence
  P2A
GATNFSLLKQAGDVEENPGP

SEQ ID NO: 30 - protein, 22 aa, Artificial Sequence
  P2A*
GSGATNFSLLKQAGDVEENPGP

SEQ ID NO: 31 - protein, 20 aa, Artificial Sequence
  E2A
QCTNYALLKLAGDVESNPGP

SEQ ID NO: 32 - protein, 23 aa, Artificial Sequence
  E2A*
GSGQCTNYALLKLAGDVESNPGP

SEQ ID NO: 33 - protein, 22 aa, Artificial Sequence
  F2A
VKQTLNFDLLKLAGDVESNPGP

SEQ ID NO: 34 - protein, 25 aa, Artificial Sequence
  F2A*
GSGVKQTLNFDLLKLAGDVESNPGP

SEQ ID NO: 35 - protein, 25 aa, Artificial Sequence
  MLS functional variant
MSVLTPLLLRGLTGSARRLPVPRAK

SEQ ID NO: 36 - protein, 61 aa, Artificial Sequence
  MLS functional variant
MQTAGALFISPALIRCCTRGLIRPVSASFLNSPVNSSKQPSYSNFPLQVARREFQTSVVSR

SEQ ID NO: 37 - protein, 47 aa, Artificial Sequence
  MLS functional variant
MLGFVGRVAAAPASGALRRLTPSASLPPAQLLLRAAPTAVHPVRDYA

SEQ ID NO: 38 - protein, 17 aa, Artificial Sequence
  MLS functional variant
MLRAAARFGPRLGRRLL

SEQ ID NO: 39 - protein, 15 aa, Artificial Sequence
  MLS functional variant
RSGIIKASRVLYRQM

SEQ ID NO: 40 - DNA, 37 bp, Artificial Sequence
  HH (Hammerhead) ribozyme
ctgatgagtccgtgaggacgaaacgagtaagctcgtc

SEQ ID NO: 41 - DNA, 68 bp, Artificial Sequence
  hepatitis delta virus (HDV) ribozyme
ggccggcatggtcccagcctcctcgctggcgccggctgggcaacatgcttcggcatggcgaatgggac

SEQ ID NO: 42 - DNA, 13 bp, Artificial Sequence
  Kozak sequence
gccgccrccaugg